(19) United States
(12) Patent Application Publication
(10) Pub. No.: US 2019/0111269 A1
(43) Pub. Date: Apr. 18, 2019

(54) SEMICONDUCTOR INTEGRATED CIRCUIT, PACEMAKER PULSE DETECTION APPARATUS, AND PACEMAKER PULSE DETECTION METHOD

(71) Applicant: SOCIONEXT INC., Yokohama (JP)

(72) Inventors: Ryusuke Kurachi, Yokohama (JP); Tatsuya Ide, Yokohama (JP); Masato Yoshioka, Yokohama (JP); Masaya Tamamura, Yokohama (JP); Amane Inoue, Yokohama (JP)

(21) Appl. No.: 16/151,990

(22) Filed: Oct. 4, 2018

(30) Foreign Application Priority Data
Oct. 16, 2017 (JP) 2017-200060

(51) Int. Cl. A61N 1/37

(52) U.S. Cl. CPC A61N 1/371; A61N 1/3704

(57) ABSTRACT

An acquisition circuit obtains an electrocardiogram waveform on which a pulse signal of a pacemaker is superimposed. A detector circuit performs a filtering process on the electrocardiogram waveform at a cutoff frequency that is determined according to a control signal, and detects a pulse signal on the basis of the result of the filtering process. A control circuit detects, a plurality of times, an occurrence start time of a QRS complex from the electrocardiogram waveform obtained by the acquisition circuit, and detects a QRS duration, and then determines a predicted occurrence start time of the next QRS complex on the basis of the detected occurrence start times of the QRS complexes, and supplies, to the detector circuit, a control signal for instructing to increase the cutoff frequency from a first value to a second value during a period based on the QRS duration from the predicted occurrence start time.

### CROSS-REFERENCE TO RELATED APPLICATION

This application is based upon and claims the benefit of priority of the prior Japanese Patent Application No. 2017-200060, filed on Oct. 16, 2017, the entire contents of which are incorporated herein by reference.

### FIELD

The embodiments discussed herein relate to a semiconductor integrated circuit, a pacemaker pulse detection apparatus, and a pacemaker pulse detection method.

### BACKGROUND

When an electrocardiogram is performed on a patient with an implanted pacemaker, a pulse signal of the pacemaker (hereinafter, referred to as pacemaker pulse) superimposed on an electrocardiogram waveform is detected to confirm if the pacemaker inside the patient operates properly.

In order to improve the accuracy of pacemaker pulse detection, the sampling rate for analog-to-digital (AD) conversion of the electrocardiogram waveform is increased because the pacemaker pulse has higher frequency components than the electrocardiogram waveform. However, to increase the sampling rate leads to consuming more power. To deal with this, a dedicated circuit with a high-pass filter for passing only pacemaker pulses with high frequency components is conventionally provided to detect the pacemaker pulses, separately from a circuit for detecting an electrocardiogram waveform.

Please see, for example, Japanese Published Unexamined Utility Model Application No. 60-180406.

Japanese Laid-open Patent Publication No. 04-336032.

Japanese Laid-open Patent Publication No. 2009-240623.

By the way, with advances in pacemakers, pacemaker pulses become smaller. If the cutoff frequency of a high-pass filter is set higher in order to remove an electrocardiogram waveform more accurately and thereby to prevent enormous detection, such a small pacemaker pulse has smaller amplitude and narrower width after passing through the high-pass filter, which may result in failing to detect the pacemaker pulse. If the cutoff frequency is set lower, the high-pass filter may fail to achieve sufficient removal of an electrocardiogram waveform, so that part of the electrocardiogram waveform may be erroneously detected as a pacemaker pulse. In view of this, it is an object to improve the accuracy of pacemaker pulse detection.

### SUMMARY

According to one aspect, there is provided a semiconductor integrated circuit including: an acquisition circuit configured to obtain an electrocardiogram waveform on which a pulse signal of a pacemaker is superimposed; a first detector circuit configured to perform a filtering process on the electrocardiogram waveform at a cutoff frequency that is determined according to a control signal and detect the pulse signal based on a result of the filtering process; and a control circuit configured to detect, a plurality of times, an occurrence start time of a QRS complex from the electrocardiogram waveform obtained by the acquisition circuit, and detect a QRS duration, and then determine a predicted occurrence start time of a next QRS complex based on detected occurrence start times, and supply, to the first detector circuit, the control signal for instructing to increase the cutoff frequency from a first value to a second value during a period based on the QRS duration from the predicted occurrence start time.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 illustrates an example of a pacemaker pulse detection apparatus and a semiconductor integrated circuit according to a first embodiment;

FIG. 2 illustrates an example of erroneous detection that occurs in the case where a cutoff frequency is set relatively low during the course of a filtering process;

FIG. 3 illustrates an example of a pacemaker pulse detection apparatus and a semiconductor integrated circuit according to a second embodiment;

FIG. 4 is a flowchart illustrating a process of detecting pacemaker pulses;

FIG. 5 illustrates an example of a process of sending information about an electrocardiogram waveform and pacemaker pulses; and

FIG. 6 illustrates an example of a pacemaker pulse detection apparatus and a semiconductor integrated circuit according to a third embodiment.

### DESCRIPTION OF EMBODIMENTS

Hereinafter, preferred embodiments will be described with reference to the accompanying drawings.

### First Embodiment

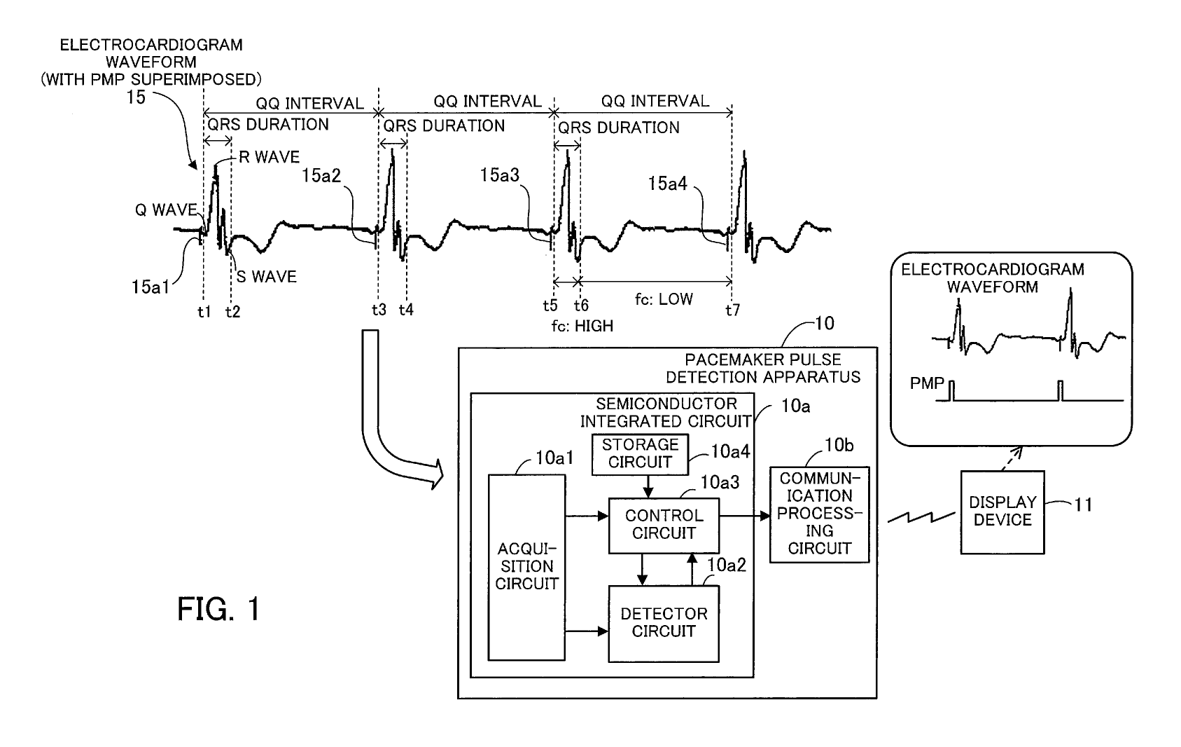

FIG. 1 illustrates an example of a pacemaker pulse detection apparatus and a semiconductor integrated circuit according to a first embodiment.

The pacemaker pulse detection apparatus 10 obtains electrocardiogram waveforms (with pacemaker pulses superimposed) from a plurality of electrodes attached to the body of a patient with an implanted pacemaker, and detects the pacemaker pulses. FIG. 1 illustrates an example of an electrocardiogram waveform 15. In the electrocardiogram waveform 15, pulses 15a1, 15a2, 15a3, and 15a4 are pacemaker pulses. In this connection, the electrocardiogram waveform 15 includes a plurality of waves, namely Q wave, R wave, and S wave. A Q wave, an R wave, and an S wave are collectively called a QRS complex. As illustrated in FIG. 1, triggered by a pacemaker pulse, a QRS complex occurs immediately after the pacemaker pulse is generated.

Note that the electrodes attached to the patient's body and so on are not illustrated in FIG. 1.

The pacemaker pulse detection apparatus 10 includes a semiconductor integrated circuit 10a and a communication processing circuit 10b.

The semiconductor integrated circuit 10a includes an acquisition circuit 10al, a detector circuit 10a2, a control circuit 10a3, and a storage circuit 10a4.

The acquisition circuit 10a1 obtains the above-described electrocardiogram waveform 15. The acquisition circuit 10a1 is an input buffer circuit, for example.

The detector circuit 10a2 performs a filtering process on the obtained electrocardiogram waveform 15 at a cutoff frequency and detects pacemaker pulses on the basis of the result of the filtering process. The cutoff frequency is determined according to a control signal supplied from the control circuit 10a3, as will be described later. For example, the detector circuit 10a2 is implemented by using a high-pass filter, a comparator, a counter, and others (an example of the detector circuit using these circuits will be described in a second embodiment).

The control circuit 10a3 detects, a plurality of times, an occurrence start time of a QRS complex from the electrocardiogram waveform 15 obtained by the acquisition circuit 10al, and also detects a QRS duration. Although the electrocardiogram waveform 15 is supplied as a digital value to the control circuit 10a3 after being subjected to an amplification process and AD conversion process, FIG. 1 does not illustrate an amplifier unit and others. The occurrence start time of a QRS complex is the occurrence start time of a Q wave (or the time the Q wave is detected). The QRS duration indicates a period of time between the occurrence start time of the Q wave and the occurrence end time of the S wave. In this connection, a period of time between when the Q wave is detected and when the S wave is detected may be taken as the QRS duration.

In addition, the control circuit 10a3 determines a predicted occurrence start time of the next QRS complex on the basis of the detected occurrence start times of the QRS complexes. The control circuit 10a3 then supplies, to the detector circuit 10a2, a control signal for instructing to increase the cutoff frequency from a first value to a second value during a period based on the QRS duration from the predicted occurrence start time. For example, the first value is an initial value of the cutoff frequency and is a relatively low cutoff frequency for passing waves with relatively high frequencies, like R waves, as well as pacemaker pulses. The second value is a high cutoff frequency for removing waves with relatively high frequencies, like R waves.

For example, the control circuit 10a3 is a processor, such as a Central Processing Unit (CPU) or a Digital Signal Processor (DSP), or a set of multiple processors (may be called a multiprocessor). In this connection, the control circuit 10a3 may include an Application-Specific Integrated Circuit (ASIC), a Field Programmable Gate Array (FPGA), or another application-specific electronic circuit. The control circuit 10a3 runs a program stored in the storage circuit 10a4 using various data stored in the storage circuit 10a4.

For example, the storage circuit 10a4 includes a volatile storage device, such as a Random Access Memory (RAM), and a non-volatile storage device, such as a flash memory or an Electrically Erasable Programmable Read-Only Memory (EEPROM). For example, a program stored in the non-volatile storage device may be loaded to the volatile storage device and then executed, under the control of the control circuit 10a3. The programs include programs for performing a process of detecting the occurrence start time of a QRS complex and a QRS duration on the basis of the characteristics of an electrocardiogram waveform, a process of controlling a cutoff frequency, and a process of controlling the communication processing circuit 10b.

The communication processing circuit 10b performs wired or radio communication with an external device, which is provided outside the pacemaker pulse detection apparatus 10. FIG. 1 illustrates an example where the communication processing circuit 10b performs radio communication to send information about an electrocardiogram waveform and detected pacemaker pulses to a display device 11 (for example, a tablet terminal).

In the case where plural types of electrocardiogram waveforms are obtained, an additional circuit is provided to select one of these types and supply the selected electrocardiogram waveform to the detector circuit 10a2 and the control circuit 10a3. However, such a circuit is not illustrated in FIG. 1 for the sake of simple explanation, but will be described in the second embodiment.

The following describes how the pacemaker pulse detection apparatus 10 and semiconductor integrated circuit 10a of the first embodiment operate.

When the acquisition circuit 10a1 obtains the electrocardiogram waveform 15 illustrated in FIG. 1, the control circuit 10a3 detects, a plurality of times, the occurrence start time of a QRS complex and a QRS duration. For example, the control circuit 10a3 detects the occurrence start time of a QRS complex at time t1, and at time t2 that is the occurrence end time of an S wave, calculates a period of time between time t1 and time t2 to thereby detect a QRS duration. Similarly, the control circuit 10a3 detects the occurrence start time of a QRS complex at time t3 and detects a QRS duration at time t4 that is the occurrence end time of an S wave.

For example, the control circuit 10a3 determines the occurrence start time (predicted occurrence start time) of the next QRS complex on the basis of the occurrence start time (time t3) of the currently detected QRS complex and the occurrence start time (time t1) of the previous QRS complex. The control circuit 10a3 determines the predicted occurrence start time by adding a time interval from time t1 to time t3 (hereinafter, this time interval is referred to as a QQ interval), to time t3. Referring to the example of FIG. 1, time t5 is determined as the predicted occurrence start time of the next QRS complex.

In addition, the control circuit 10a3 supplies, to the detector circuit 10a2, a control signal for instructing to set a cutoff frequency fc to be used during a period from time t5 to time t6, to higher than a value that is preset as the cutoff frequency fc to be used outside the period of QRS duration. The time t6 is when the period of the detected QRS duration passes after time t5.

Note that the control circuit 10a3 also detects an actual occurrence start time with respect to the third QRS complex from the left of FIG. 1 (in the example of FIG. 1, this time is the same as the predicted occurrence start time), and determines the predicted occurrence start time of the next QRS complex. In the example of FIG. 1, time t7 is determined as the predicted occurrence start time of the next QRS complex.

In addition, the control circuit 10a3 also detects a QRS duration with respect to the third QRS complex from the left of FIG. 1.

The control circuit 10a3 supplies, to the detector circuit 10a2, a control signal for instructing to set the cutoff frequency fc to be used during a period from time t6 to time t7, to lower than a value that is used during the period of QRS duration.

The detector circuit 10a2 changes the cutoff frequency fc according to the above-described control signal and performs a filtering process on the electrocardiogram waveform to detect pacemaker pulses. Every time the detector circuit 10a2 detects a pacemaker pulse, it notifies the control circuit 10a3 of this detection.

The control circuit 10a3 supplies, to the communication processing circuit 10b, information about the electrocardiogram waveform and the detected pacemaker pulses. For example, the communication processing circuit 10b sends the information on the electrocardiogram waveform and the detected pacemaker pulses to the display device 11 in real time. For example, the electrocardiogram waveform and detected pacemaker pulses (represented as PMP in FIG. 1) are displayed on the screen of the display device 11, as illustrated in FIG. 1.

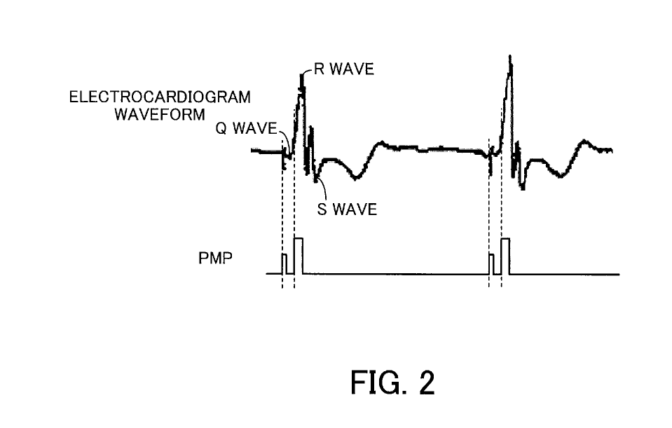

FIG. 2 illustrates an example of erroneous detection that occurs in the case where a cutoff frequency is set relatively low during the course of a filtering process.

In the case where a cutoff frequency is set relatively low during the course of a filtering process, not only pacemaker pulses are detected correctly, but also R waves may be erroneously detected as pacemaker pulses.

By contrast, the pacemaker pulse detection apparatus 10 and semiconductor integrated circuit 10a of the first embodiment set the cutoff frequency high for the filtering process performed by the detector circuit 10a2 while a QRS complex appears, as described above. By doing so, waves with relatively high frequency components, like R waves, have smaller amplitudes after the filtering process, and it is possible to reduce a risk of erroneously detecting the R waves with relatively high frequency components as pacemaker pulses.

In addition, while no QRS complex appears, the cutoff frequency is set low for the filtering process performed by the detector circuit 10a2, so that it is possible to reduce a risk of failing to detect pacemaker pulses even if the pacemaker pulses are very small.

As a result, the pacemaker pulse detection apparatus 10 and semiconductor integrated circuit 10a of the first embodiment make it possible to improve the accuracy of pacemaker pulse detection.

In addition, by determining the predicted occurrence start time of the next QRS complex and a QRS duration to be used, on the basis of a result of detecting the occurrence start time of the previous QRS complex and the QRS duration, it is possible to control the cutoff frequency at more proper timing and therefore to further improve the accuracy of pacemaker pulse detection.

In this connection, the control circuit 10a3 may be designed to obtain an average value of a plurality of QQ intervals detected and then to determine the predicted occurrence start time of the next QRS complex using the average value. In addition, the control circuit 10a3 may be designed to obtain an average value of a plurality of QRS durations detected and then to increase the cutoff frequency during a period indicated by the average value.

### Second Embodiment

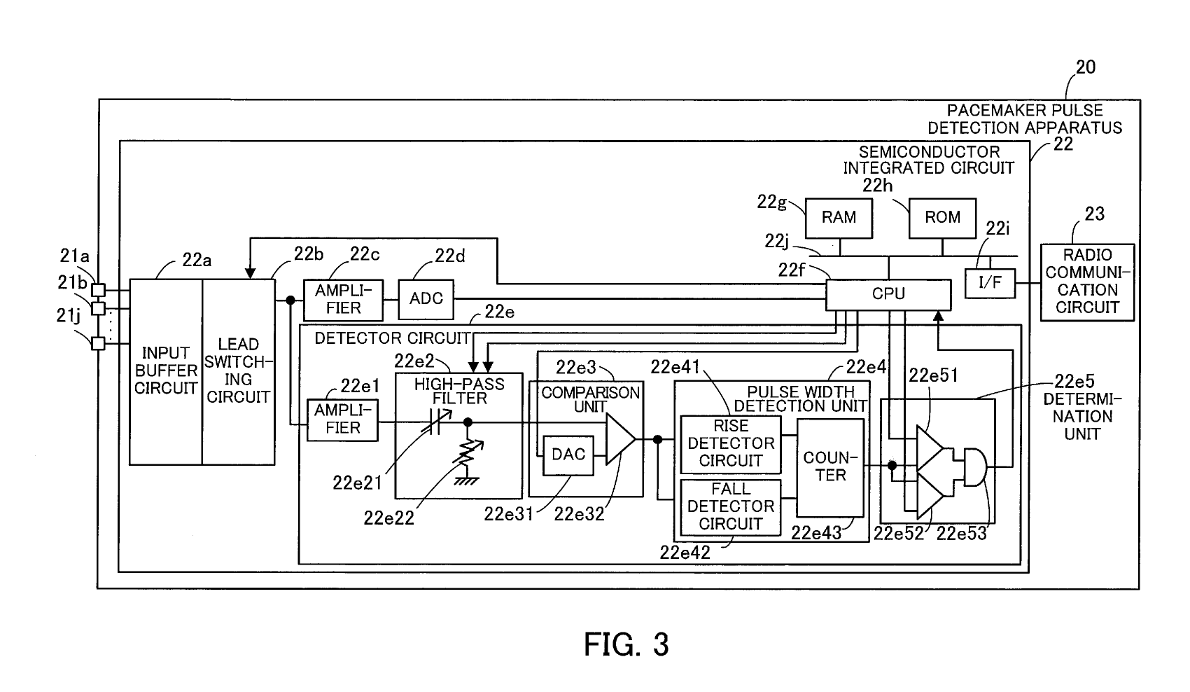

FIG. 3 illustrates an example of a pacemaker pulse detection apparatus and a semiconductor integrated circuit according to the second embodiment.

The pacemaker pulse detection apparatus 20 of the second embodiment includes input terminals 21a, 21b, . . . , 21j that are coupled to, for example, ten electrodes attached to the body of a patient with an implanted pacemaker, a semiconductor integrated circuit 22, and a radio communication circuit 23.

In the standard 12-lead electrocardiogram using ten electrodes, twelve kinds of potential differences between prescribed two electrodes are measured using four electrodes each attached to a different one of the patient's arms and legs and six electrodes attached to the patient's chest. Thereby, twelve types of electrocardiogram waveforms are obtained. In this connection, the number of electrodes is not limited to ten but a fewer or greater number of electrodes may be used as long as at least one electrocardiogram diagram is obtained.

The semiconductor integrated circuit 22 includes an input buffer circuit 22a, a lead switching circuit 22b, an amplifier 22c, an AD converter circuit (represented as “ADC” in FIG. 3) 22d, and a detector circuit 22e. The semiconductor integrated circuit 22 further includes a CPU 22f, a RAM 22g, a ROM 22h, an interface circuit (represented as “I/F” in FIG. 3) 22i, and a bus 22j.

The input buffer circuit 22a obtains and holds plural types of electrocardiogram waveforms input via the input terminals 21a to 21j. In the case of the standard 12-lead electrocardiogram using ten electrodes, the input buffer circuit 22a obtains twelve types of electrocardiogram waveforms.

The lead switching circuit 22b selects and outputs one of the plural types of electrocardiogram waveforms obtained by the input buffer circuit 22a, under the control of the CPU 22f.

The amplifier 22c amplifies and outputs the electrocardiogram waveform output by the lead switching circuit 22b.

The AD converter circuit 22d converts the electrocardiogram waveform output by the amplifier 22c into a digital value, and outputs the digital value.

The detector circuit 22e detects pacemaker pulses superimposed on the electrocardiogram waveform output by the lead switching circuit 22b. The detector circuit 22e includes an amplifier 22e1, a high-pass filter 22e2, a comparison unit 22e3, a pulse width detection unit 22e4, and a determination unit 22e5.

The amplifier 22e1 amplifies and outputs the electrocardiogram waveform output by the lead switching circuit 22b.

The high-pass filter 22e2 includes a variable capacitance element 22e21 and a variable resistance element 22e22, and performs a filtering process on the electrocardiogram waveform output by the amplifier 22e1, at a cutoff frequency determined based on the capacitance value of the variable capacitance element 22e21 and the resistance value of the variable resistance element 22e22. These capacitance value and resistance value are adjusted by the CPU 22f.

The comparison unit 22e3 includes a digital-to-analog (DA) converter circuit (represented as “DAC” in FIG. 3) 22e31 and a comparator 22e32. The DA converter circuit 22e31 converts a digital threshold value supplied from the CPU 22f into an analog value. The comparator 22e32 compares the analog threshold value with an output signal of the high-pass filter 22e2, and outputs “1” when the output signal of the high-pass filter 22e2 is greater than or equal to the threshold value, and outputs “0” when the output signal is less than the threshold value. The threshold value is adjustable according to a result of detecting pacemaker pulses. The comparison unit 22e3, which operates in this way, is able to eliminate the influences of an electrocardiogram waveform after the filtering process, which is less than the threshold value.

The pulse width detection unit 22e4 detects the pulse width of the output signal of the comparison unit 22e3. The pulse width represents a period of time during which the output signal of the high-pass filter 22e2 is greater than or equal to the threshold value.

The pulse width detection unit 22e4 includes a rise detector circuit 22e41, a fall detector circuit 22e42, and a counter 22e43. The rise detector circuit 22e41 outputs a signal indicating that a rise has been detected, when the output signal of the comparison unit 22e3 varies from “0” to “1”. The fall detector circuit 22e42 outputs a signal indicating that a fall has been detected, when the output signal of the comparison unit 22e3 varies from “1” to “0”. The counter 22e43 is given a clock signal, not illustrated. The counter 22e43 counts the number of clocks during a period from when the rise detector circuit 22e41 detects a rise to when the fall detector circuit 22e42 detects a fall, and outputs the count value as a pulse width.

The determination unit 22e5 outputs a signal indicating that a pacemaker pulse has been detected, when the pulse width output from the pulse width detection unit 22e4 falls between lower and upper limits of pulse width. These limits are given by the CPU 22f. The determination unit 22e5 includes comparators 22e51 and 22e52 and an AND (logical conjunction) circuit 22e53. The comparator 22e51 compares the pulse width output from the pulse width detection unit 22e4 with the upper limit of pulse width given by the CPU 22f, and outputs “1” when the pulse width is less than or equal to the upper limit, and outputs “0” when the pulse width exceeds the upper limit. The comparator 22e52 compares the pulse width output from the pulse width detection unit 22e4 with the lower limit of pulse width given by the CPU 22f, and outputs “1” when the pulse width is greater than or equal to the lower limit, and outputs “0” when the pulse width is less than the lower limit. The AND circuit 22e53 outputs “1” as a signal indicating that the pacemaker pulse has been detected, when the comparators 22e51 and 22e52 both output “1”. The AND circuit 22e53 outputs “0” when at least one of the comparators 22e51 and 22e52 outputs “0”.

Note that the upper and lower limits of pulse width are set according to the pulse widths of pacemaker pulses output from the pacemaker implanted into the patient. The upper and lower limits are adjustable according to a result of detecting the pacemaker pulses.

The CPU 22f loads at least part of a program and data from the ROM 22h to the RAM 22g via the bus 22j and runs the program. The CPU 22f runs the program to change the cutoff frequency of the high-pass filter 22e2 by adjusting the above-described capacitance value and resistance value.

The RAM 22g is a volatile semiconductor memory for temporarily storing programs to be run by the CPU 22f and data to be used by the CPU 22f in operation.

The ROM 22h is a non-volatile storage device for storing programs to be run by the CPU 22f and various data.

The interface circuit 22i communicates information with the radio communication circuit 23 under the control of the CPU 22f.

The radio communication circuit 23 performs radio communication to exchange information with an external device (for example, a tablet terminal) provided outside the pacemaker pulse detection apparatus 20.

The following describes how the pacemaker pulse detection apparatus 20 and semiconductor integrated circuit 22 of the second embodiment detect pacemaker pulses.

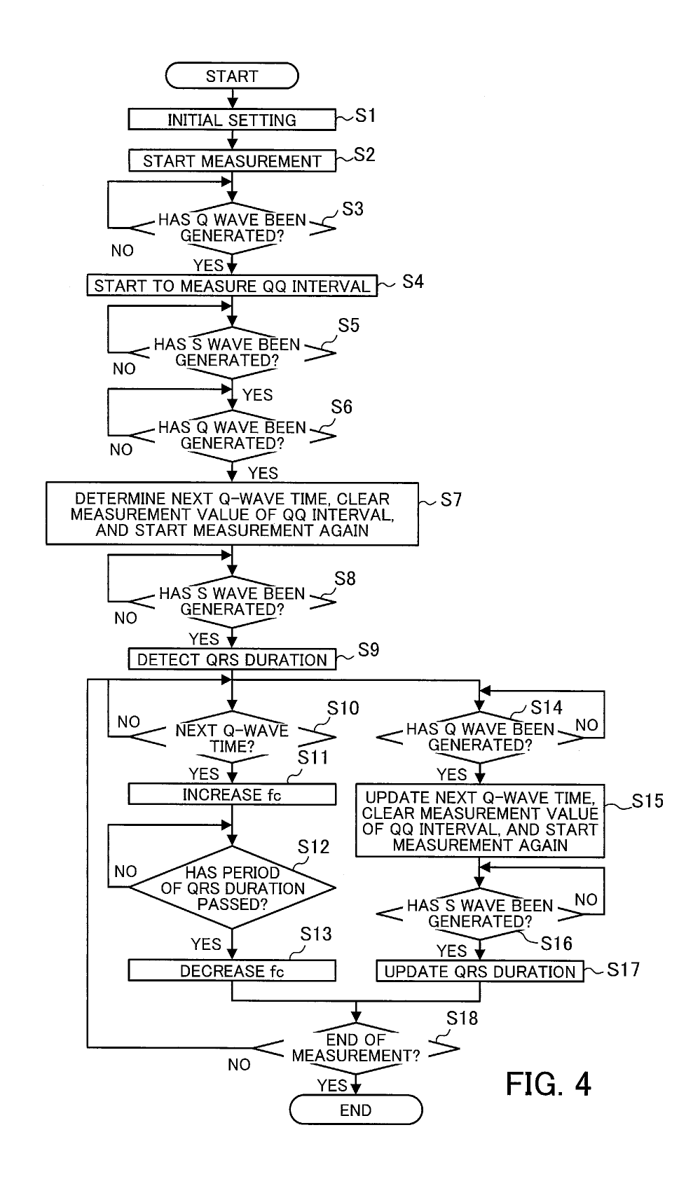

FIG. 4 is a flowchart illustrating a process of detecting pacemaker pulses.

First, the CPU 22f performs initial setting (step S1). At step S1, the CPU 22f adjusts the capacitance value of the variable capacitance element 22e21 and the resistance value of the variable resistance element 22e22, and sets the cutoff frequency of the high-pass filter 22e2 to the initial value. The CPU 22f also sets the threshold value to be given to the comparison unit 22e3 and the lower and upper limits of pulse width to be given to the determination unit 22e5. The CPU 22f also gives a signal indicating a type of electrocardiogram waveform to be output among plural types of electrocardiogram waveforms, to the lead switching circuit 22b.

When measurement of an electrocardiogram waveform starts (step S2), the CPU 22f determines whether a Q wave has been generated, on the basis of digital values of the electrocardiogram waveform given via the input buffer circuit 22a, lead switching circuit 22b, amplifier 22c, and AD converter circuit 22d (step S3). Step S3 is repeated until the generation of a Q wave is detected.

When detecting the generation of a Q wave, the CPU 22f starts to measure a QQ interval using a timer, for example (step S4).

Then, the CPU 22f determines whether an S wave has been generated, on the basis of digital values of the electrocardiogram waveform (step S5). Step S5 is repeated until the generation of an S wave is detected.

After detecting the generation of an S wave, the CPU 22f determines whether a Q wave has been generated (step S6). Step S6 is repeated until the generation of a Q wave is detected.

After detecting the generation of a Q wave, the CPU 22f determines the next Q-wave time (corresponding to the predicted occurrence start time of the next QRS complex, which is described earlier), clears the measurement value of the QQ interval, and starts the measurement again (step S7). The next Q-wave time is determined by adding the currently obtained measurement value of the QQ interval to the time when the Q wave was detected at step S6.

Then, the CPU 22f determines whether an S wave has been generated, on the basis of digital values of the electrocardiogram waveform (step S8). Step S8 is repeated until the generation of an S wave is detected.

After detecting the generation of an S wave, the CPU 22f detects a QRS duration (step S9). The CPU 22f detects the QRS duration by calculating a period of time between when a Q wave was detected at step S6 and when an S wave was detected at step S8.

After that, for example, steps S10 to S13 and steps S14 to S17 are executed in parallel, as described below.

The CPU 22f determines whether the next Q-wave time determined at step S7 has come (step S10). If the next Q-wave time has not come, step S10 (or step S14) is repeated, and then the successive steps are executed.

If the next Q-wave time has come, the CPU 22f adjusts the capacitance value of the variable capacitance element 22e21 and the resistance value of the variable resistance element 22e22 in order to set the cutoff frequency fc higher than the initial value (step S11).

After that, the CPU 22f determines whether the period of the QRS duration detected at step S9 (or updated at step S17 as described later) has passed (step S12). If the period of the QRS duration has not passed, step S12 is repeated.

If the period of the QRS duration has passed, the CPU 22f adjusts the capacitance value of the variable capacitance element 22e21 and the resistance value of the variable resistance element 22e22 in order to set the cutoff frequency fc lower (for example, back to the initial value) (step S13).

After step S9, the CPU 22f determines whether a Q wave has been generated (step S14). Step S14 is repeated until the generation of a Q wave is detected.

After detecting the generation of a Q wave, the CPU 22f updates the next Q-wave time, clears the measurement value of the QQ interval, and starts the measurement again (step S15).

After that, the CPU 22f determines whether an S wave has been generated, on the basis of digital values of the electrocardiogram waveform (step S16). Step S16 is repeated until the generation of an S wave is detected.

After detecting the generation of an S wave, the CPU 22f updates the QRS duration (step S17).

After step S13 or S17, step S10 or S14 and the subsequent steps are repeated if the measurement of the electrocardiogram waveform has not ended (No at step S18). If the measurement of the electrocardiogram waveform has ended (for example, if an instruction to end the measurement is received from an external device) (Yes at step S18), the CPU 22f ends the pacemaker pulse detection process.

In the above processing of the CPU 22f, the cutoff frequency fc of the high-pass filter 22e2 is set higher while a QRS complex appears. Therefore, waves with relatively high frequencies, like R waves, have smaller amplitudes after the filtering process, thereby reducing a risk of erroneously detecting such waves as pacemaker pulses.

In addition, the cutoff frequency of the high-pass filter 22e2 is set lower while no QRS complex appears, thereby reducing a risk of failing to detect pacemaker pulses even if the pacemaker pulses are very small.

As a result, the pacemaker pulse detection apparatus 20 and semiconductor integrated circuit 22 of the second embodiment make it possible to improve the accuracy of pacemaker pulse detection.

Note that the order of steps in FIG. 4 is just an example and may be changed where appropriate. For example, in the above example, steps S10 to S13 and steps S14 to S17 are executed in parallel. Alternatively, the determination steps S10 and S14 may be executed alternately, at different times.

By the way, a signal indicating whether a pacemaker pulse has been detected or not is supplied from the determination unit 22e5 to the CPU 22f. The CPU 22f gives information about the pacemaker pulse, together with information about the electrocardiogram waveform, to the radio communication circuit 23 via the interface circuit 22i.

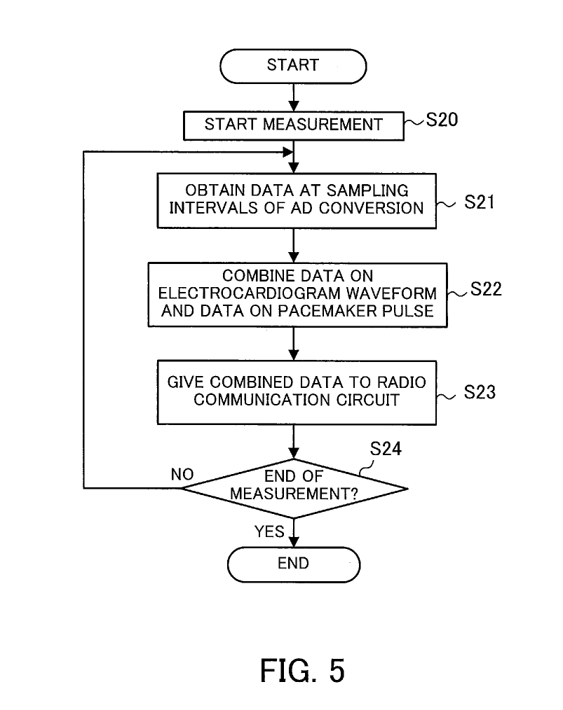

FIG. 5 illustrates an example of a process of sending information about an electrocardiogram waveform and pacemaker pulses.

When measurement of an electrocardiogram waveform starts (step S20), the CPU 22f obtains two types of data: a digital value of an electrocardiogram waveform, and a signal indicating whether a pacemaker pulse has been detected, supplied from the determination unit 22e5, at sampling intervals of AD conversion (step S21).

Then, the CPU 22f combines these two types of data (step S22) and gives the resultant to the radio communication circuit 23 (step S23).

After that, if the measurement of the electrocardiogram waveform has not ended (No at step S24), step S21 and the subsequent steps are repeated. If the measurement of the electrocardiogram waveform has ended (Yes at step S24), the CPU 22f ends the sending process.

With the above process, data combining a digital value of an electrocardiogram waveform and information indicating whether a pacemaker pulse has been detected is sent to the radio communication circuit 23 at sampling intervals of the AD conversion. Then, the radio communication circuit 23 sends the combined data to an external device. For example, the radio communication circuit 23 sends the combined data to the display device 11 illustrated in FIG. 1, and the display device 11 displays the electrocardiogram waveform and pacemaker pulse on its screen in real time.

Note that the operator of the pacemaker pulse detection apparatus 20 may confirm the display screen of the display device 11 and enter a command to change the cutoff frequency, the threshold, or the upper or lower limit of pulse width to the display device 11 according to the detected pacemaker pulses. In this case, the command is transferred to the semiconductor integrated circuit 22, and the CPU 22f changes the cutoff frequency, the threshold, or the upper or lower limit of pulse width accordingly. In addition, the operator of the pacemaker pulse detection apparatus 20 may confirm the display screen of the display device 11 and enter a command to display an electrocardiogram waveform other than the electrocardiogram waveform currently displayed on the display screen, among twelve types of electrocardiogram waveforms, to the display device 11. In this case, the command is transferred to the semiconductor integrated circuit 22, and the CPU 22f instructs the lead switching circuit 22b to output the requested electrocardiogram waveform according to the command. The above adjustments make it possible to further improve the accuracy of pacemaker pulse detection.

### Third Embodiment

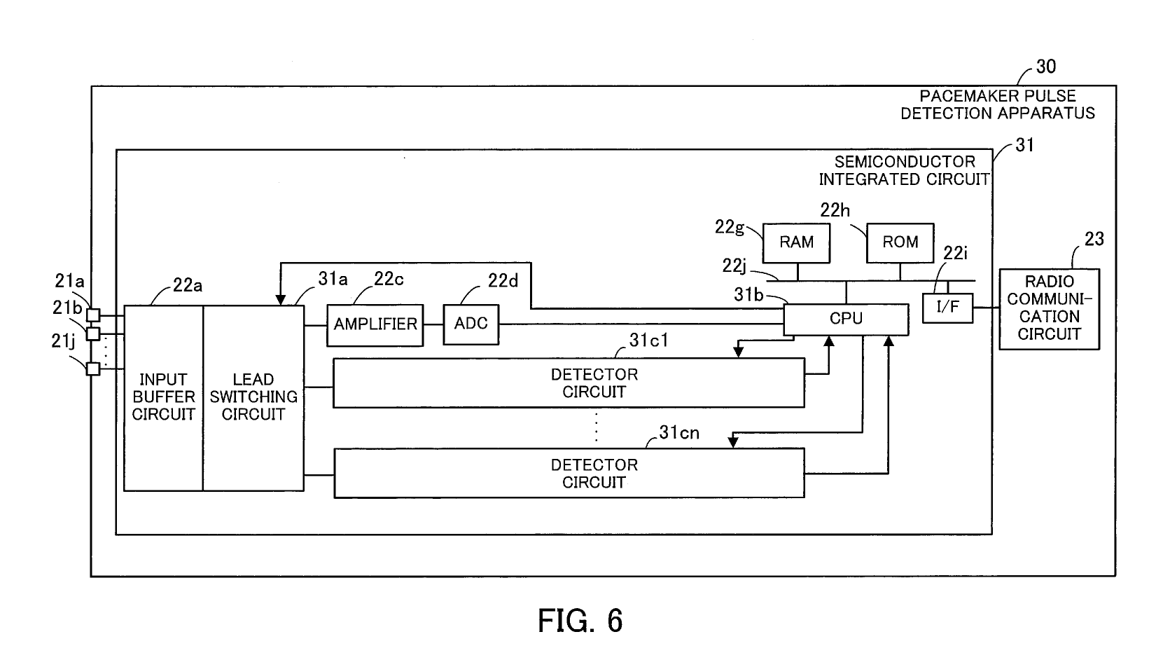

FIG. 6 illustrates an example of a pacemaker pulse detection apparatus and a semiconductor integrated circuit according to a third embodiment. The constitutional elements that are identical to those of the pacemaker pulse detection apparatus 20 of the second embodiment of FIG. 3 will be given the same reference numerals as the corresponding constitutional elements of the second embodiment.

In the pacemaker pulse detection apparatus of the third embodiment, a lead switching circuit 31a of a semiconductor integrated circuit 31 obtains n (n≥2) types of electrocardiogram waveforms among plural different types of electrocardiogram waveforms, under the control of a CPU 31b. The plural different types of electrocardiogram waveforms are obtained by different pairs of electrodes attached to a patient's body. The lead switching circuit 31a supplies the obtained n types of electrocardiogram waveforms to n detector circuits 31c1 to 31cn. In addition, the lead switching circuit 31a supplies one of the plural types of electrocardiogram waveforms to an amplifier 22c, under the control of the CPU 31b.

The detector circuits 31c1 to 31cn each have the same elements as the detector circuit 22e of FIG. 3. Therefore, the CPU 31b receives signals each indicating whether a pacemaker pulse has been detected from one of the n types of electrocardiogram waveforms, from the detector circuits 31c1 to 31cn.

The CPU 31b determines based on the signals received from the detector circuits 31c1 to 31cn whether a pacemaker pulse has been detected. In the case where, at a certain time, signals from some of the detector circuits 31c1 to 31cn indicate that a pacemaker pulse has been detected and signals from the others of the detector circuits 31c1 to 31cn indicate that no pacemaker pulse has been detected, the CPU 31b determines by majority whether a pacemaker pulse has been detected.

As a result, it is possible to further improve the accuracy of pacemaker pulse detection.

According to one aspect, it is possible to improve the accuracy of pacemaker pulse detection.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A semiconductor integrated circuit comprising:
an acquisition circuit configured to obtain an electrocardiogram waveform on which a pulse signal of a pacemaker is superimposed;
a first detector circuit configured to perform a filtering process on the electrocardiogram waveform at a cutoff frequency that is determined according to a control signal and detect the pulse signal based on a result of the filtering process; and
a control circuit configured to detect, a plurality of times, an occurrence start time of a QRS complex from the electrocardiogram waveform obtained by the acquisition circuit, and detect a QRS duration, and then determine a predicted occurrence start time of a next QRS complex based on detected occurrence start times, and supply, to the first detector circuit, the control signal for instructing to increase the cutoff frequency from a first value to a second value during a period based on the QRS duration from the predicted occurrence start time.

2. The semiconductor integrated circuit according to claim 1, wherein the control circuit determines the predicted occurrence start time by adding a time interval between a first occurrence start time and a second occurrence start time, among the detected occurrence start times, to the second occurrence start time, the second occurrence start time being detected next to the first occurrence start time.

3. The semiconductor integrated circuit according to claim 1, wherein the first detector circuit includes
a high-pass filter configured to perform the filtering process on the electrocardiogram waveform,
a comparison unit configured to output a result of comparing an output signal of the high-pass filter with a threshold,
a pulse width detection unit configured to detect, based on the result of the comparing, a pulse width representing a period of time during which the output signal is greater than or equal to the threshold, and
a determination unit configured to supply, to the control circuit, a signal indicating that the pulse signal has been detected, when the pulse width falls between a lower limit and an upper limit.

4. The semiconductor integrated circuit according to claim 1, further comprising at least one second detector circuit having an identical function to the first detector circuit,
wherein the acquisition circuit obtains plural different types of electrocardiogram waveforms on which the pulse signal is superimposed,
wherein each of the first detector circuit and the at least one second detector circuit performs the filtering process on one of the plural different types of electrocardiogram waveforms, and detects the pulse signal based on a result of the filtering process, and
wherein the control circuit determines whether the pulse signal has been generated, based on results of detecting the pulse signal, which are respectively output from the first detector circuit and the at least one second detector circuit.

5. A pacemaker pulse detection apparatus comprising:
a semiconductor integrated circuit; and
a communication processing circuit,
wherein the semiconductor integrated circuit includes
an acquisition circuit configured to obtain an electrocardiogram waveform on which a pulse signal of a pacemaker is superimposed,
a detector circuit configured to perform a filtering process on the electrocardiogram waveform at a cutoff frequency that is determined according to a control signal and detect the pulse signal based on a result of the filtering process, and
a control circuit configured to detect, a plurality of times, an occurrence start time of a QRS complex from the electrocardiogram waveform obtained by the acquisition circuit, and detect a QRS duration, and then determine a predicted occurrence start time of a next QRS complex based on detected occurrence start times, and supply, to the detector circuit, the control signal for instructing to increase the cutoff frequency from a first value to a second value during a period based on the QRS duration from the predicted occurrence start time, and
wherein the communication processing circuit is configured to send information on the electrocardiogram waveform obtained by the acquisition circuit and the pulse signal detected by the detector circuit.

6. A pacemaker pulse detection method comprising:
obtaining, by an acquisition circuit, an electrocardiogram waveform on which a pulse signal of a pacemaker is superimposed;
performing, by a detector circuit, a filtering process on the electrocardiogram waveform at a cutoff frequency that is determined according to a control signal and detecting the pulse signal based on a result of the filtering process; and
detecting, by a control circuit, a plurality of times, an occurrence start time of a QRS complex from the electrocardiogram waveform obtained by the acquisition circuit, and detecting a QRS duration, and then determining a predicted occurrence start time of a next QRS complex based on detected occurrence start times, and supplying, to the detector circuit, the control signal for instructing to increase the cutoff frequency from a first value to a second value during a period based on the QRS duration from the predicted occurrence start time.
